(19) United States
(12) Patent Application Publication
(10) Pub. No.: US 2024/0285428 A1
(43) Pub. Date: Aug. 29, 2024

(54) SYSTEM AND METHOD FOR DYNAMIC TEMPERATURE CONTROL OF OCULAR ENVIRONMENT DURING OCULAR SURGERY

(71) Applicant: ThermOptik LLC, Baltimore, MD (US)

(72) Inventors: Rupsa B. ACHARYA, Saratoga, CA (US); Sarah KHAN, New York, NY (US); Eli B. LEVENSHUS, Norwood, NJ (US); Daniel S. WANG, Fresno, CA (US); Gwyneth W. ALEXANDER, Putney, VT (US); Rishima MUKHERJEE, Wilsonville, OR (US); Erick E. ROCHER, Orlando, FL (US); Minyoung PARK, New York, NY (US); Andres T. AGUIRRE, Miami, FL (US); James J. PITINGOLO, Oceanside, CA (US); Pratyush Sai R. RAYASAM, West Des Moines, IA (US)

(21) Appl. No.: 18/115,391

(22) Filed: Feb. 28, 2023

(51) Int. Cl. A61F 7/00; A61F 9/007

(52) U.S. Cl. CPC A61F 7/0085; A61F 9/00745; A61F 2007/0004; A61F 2007/0054; A61F 2007/0093; A61F 2007/0096

(57) ABSTRACT

A thermal control device for an ocular surgery system is provided, including a fluid heater interface module (FHIM) having an inflow port for receiving balanced salt solution (BSS) from the ocular surgery system, an outflow port for dispensing the BSS to a probe of the ocular surgery system, and internal heat transfer channels between the inflow port and the outflow port. A heating element is disposed adjacent to the FHIM to heat the heat transfer channels in the FHIM. A temperature sensor is disposed adjacent the outflow port. A microcontroller is provided to control heat output from the heating element. A user interface is provided with an outlet temperature control adjuster. A dynamic, precise temperature control of portions of the eye via the BSS is obtained. A method utilizing the FHIM is also provided.

### BACKGROUND OF THE INVENTION

The present invention is directed to ocular surgery. More particularly, the present invention is directed temperature control of the ocular environment during ocular surgery.

As we age, we accumulate protein in our eyes that causes clouding of the lens, known as cataracts. Approximately 100 million people currently have some degree of this vision loss, and over half of Americans will develop this impairment in their lifetime. Cataracts are the leading cause of blindness worldwide. Cataracts can make necessary everyday activities dangerous. An increased risk of depression and injury leads to a 40% increased risk of mortality when cataracts go untreated. As cataracts have no intermediate, non-invasive solutions, surgery is necessary to prevent future vision loss. As the global population ages, the number of cataract cases is rapidly rising. Surgeons are unable to meet this demand, causing the backlog of cases to grow year after year. In the United States alone, 1.6 million patients are waiting to receive cataract surgery, and in India, this backlog of cases has a social burden of $13.5 billion. To meet this demand, surgical throughput must be increased.

### SUMMARY OF THE INVENTION

An immediate and addressable means to increase surgical throughput of cataract surgery is to introduce temperature control of the eye environment into cataract surgery. Temperature control of the eye optimizes parameters that impact both duration and outcome of cataract surgery and improves efficiency, increases accessibility of care, and reduces postoperative complications. It will allow surgeons to avoid time-intensive bottlenecks in the procedure. The present invention decreases time per surgery and decreases the time surgeons spend handling post-operative care while improving patient outcomes.

For example, an intraocular lens (IOL) is a lens implanted in the eye as part of a treatment for cataracts. Once inserted, IOLs must unfold inside the eye before a surgeon can proceed with the surgery. Literature and benchtop studies in vitro have demonstrated a clear temperature dependence linking warmer temperatures to faster IOL unfurling. The significant reduction of surgical time allows surgical centers to complete more cataract surgeries per day.

Due to their material properties, low temperatures are associated with exponentially high unfurling times of IOLs. Thus, warmed IOLs save surgeons up to a minute per surgery, which for standard procedures is 10% of the duration. Additionally, materials used in surgery are easier to remove at higher temperatures. One example is ophthalmic viscoelastic device (OVD), a gel used to protect the delicate structures of the eye, that must be removed at the end of the procedure. At higher temperatures, this gel is more fluid and cohesive, and thus easier to remove. This reduces average surgical speed in two ways: through direct reduction of OVD removal time and decreased risk of posterior capsular rupture, a complication associated with OVD removal that adds up to 45 minutes of procedural time. In addition, warmer irrigation temperatures can reduce postoperative visits. Retained OVD is associated with postoperative intraocular pressure (IOP) spikes which lead to swelling, requiring follow-up visits.

The ocular surgery thermal control device and method of the present invention give surgeons control of the temperature of the irrigating lavage in real-time, and in a quantitative and precise manner.

The device and method of the present invention involves a heating and cooling element interfaced with balanced salt solution (BSS) tubing, and dynamically warming the BSS before it passes into the eye. The device integrates, but does not interfere with, current features of the operating room (OR) workflow such as fluidics control. Surgical technicians will easily reset the device for the next procedure by replacing the contaminated disposable portions of the device while maintaining the sterility of the heating element, encapsulation, and user interface. The device will be operated by surgical technicians and cataract surgeons via a screen on its surface and dials on its side. The device is designed to warm BSS to physiological temperature in a timeframe feasible for use in surgery.

The device of the present invention warms the entire eye environment by warming BSS, integrates into the existing workflow, and, unlike similar technology in parallel spaces, maintains precise fluidic control in addition to introducing temperature control. Existing competitors consist of makeshift solutions used by operating room staff. Body heat is commonly used, by placing the fluid or material under the armpit or against the stomach. Sometimes warming chambers for blankets or warmed blankets are used. However, these solutions introduce a significant danger of contamination or damage to the materials and warm the items inconsistently and unreliably. The device has the added benefit of dynamic modification, allowing the surgeon to warm particular steps of the procedure. This is beneficial, because cooler temperatures during phacoemulsification may reduce postoperative inflammation.

In the present invention, a thermal control device for an ocular surgery system is provided which includes a fluid heater interface module (FHIM). The FHIM has an inflow port for receiving balanced salt solution (BSS) from a BSS container of theocular surgery system, and an outflow port for dispensing the BSS to a probe of the ocular surgery system. The FHIM further includes internal heat transfer channels between the inflow port and the outflow port. At least one heating element is provided to heat the heat transfer channels in the FHIM. At least one temperature sensor is disposed adjacent the outflow port in the FHIM. A microcontroller is provided to control heat output from the heating element. Finally, a user interface includes an outlet temperature adjuster. Dynamic, precise temperature control of portions of the eye via the BSS is obtained.

A MOSFET driver may be provided to control the heating element. A power supply may be provided to provide power to the microcontroller and/or the MOSFET driver. The microcontroller may provide proportional-integral-derivative (PID) control to the control temperature of BSS exiting the outflow port. The user interface may provide for dynamic and adjustable temperature control by an ocular surgeon during ocular surgery. Temperature of BSS is displayed on the user interface in real time. The FHIM may be designed for single use.

A method for thermal control of balanced salt solution (BSS) during ocular surgery is also provided. The method first includes the step of providing a fluid heater interface module (FHIM) comprising an inflow port and an outflow port, the inflow port for receiving balanced salt solution (BSS) from an ocular surgery system, an outflow port for dispensing the BSS to a probe of the ocular surgery system, and a heating element. The method further includes the steps of heating the BSS by providing power to a heating element for heating the FHIM, controlling a temperature of the BSS (using, for example, proportional-integral-derivative control) exiting the outlet port and providing the controlled temperature BSS to an ocular surgery probe. Dynamic, precise temperature control of portions of the eye and the BSS is obtained.

### BRIEF DESCRIPTION OF THE DRAWINGS

FIG. 1 is a simplified view of a typical ocular surgery using an ocular surgery thermal control device in accordance with an exemplary embodiment of the present invention.

FIG. 2 is simplified block diagram of an ocular surgery thermal control device in accordance with an exemplary embodiment of the present invention.

FIG. 3A is a simplified exploded isometric view of the ocular surgery thermal control device of FIG. 2.

FIG. 3B is a simplified isometric view of the ocular surgery thermal control device of FIG. 2.

FIG. 4 is simplified isometric view of a fluid-heater interface module (FHIM) of the ocular surgery thermal control device of FIG. 2.

FIG. 5 is a heating element (in the form a printed circuit board) of the ocular surgery thermal control device of FIG. 2.

FIG. 6A is a simplified block diagram of elements related to a failure mode for the ocular surgery thermal control device of FIG. 2.

FIG. 6B is a simplified block diagram of secondary elements related to a failure mode for the ocular surgery thermal control device of FIG. 2.

### DETAILED DESCRIPTION

The present invention is directed to a medical device and method to assist in eliminating the growing backlog of cataract surgery cases through real-time dynamic temperature control of the ocular environment during cataract surgery. The device and method increase provider efficiency thereby improve patient outcomes, and increases accessibility to clear vision. The device and method provide for reduction of surgical time and post-operative complications.

The ocular surgery thermal control device and method of the present invention introduces precise, real-time control of temperature of the eye environment. The device and method easily integrate into typical surgical workflows, with little to no additional training required for use by scrub technicians. A disposable component maintains the strict sterility requirements of the operating room, while the device and method are compatible with systems maintaining precise pressure control of the eye. The device and method provide precise, dynamic temperature control while staying safe and sterile for patients and operating room (OR) staff. Studies in vitro have demonstrated a clear temperature dependency linking warmer temperatures to faster unfurling for acrylic hydrophobic lenses, a crucial step in cataract surgery. Additionally, increased intraocular temperature will aid in the removal of ophthalmic viscoelastic device (OVD), a gel inserted into the eye during surgery, reducing the occurrence of intraocular pressure (IOP) spikes and edema in the days following cataract surgery and risk of serious complications associated with OVD removal, such as capsular bag rupture. Both of these steps are bottlenecks in surgery, and removing them via temperature control will increase surgical efficiency by 15-20%. However, some steps of cataract surgery require a cold eye environment for optimal outcomes. The device and method of the present invention will allow for real-time heating and cooling to achieve the optimal temperature for each surgical step. Thus, the device and method of the present invention will increase the number of surgeries performed per day, reduce the number of postoperative visits required per patient on average, and restore clear vision to patients faster.

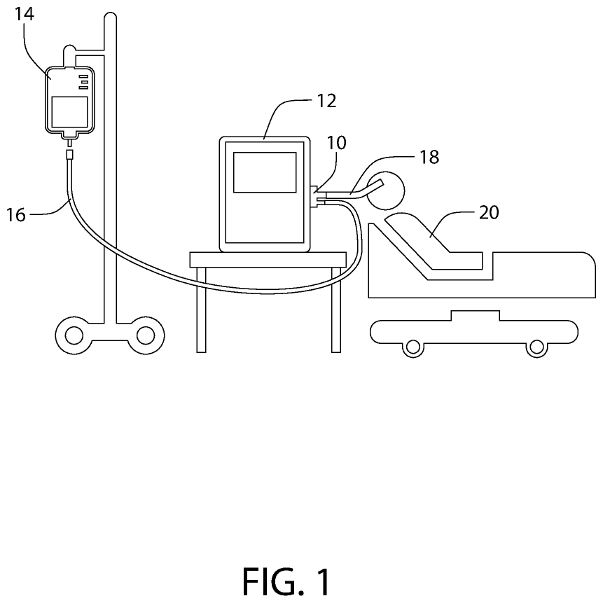

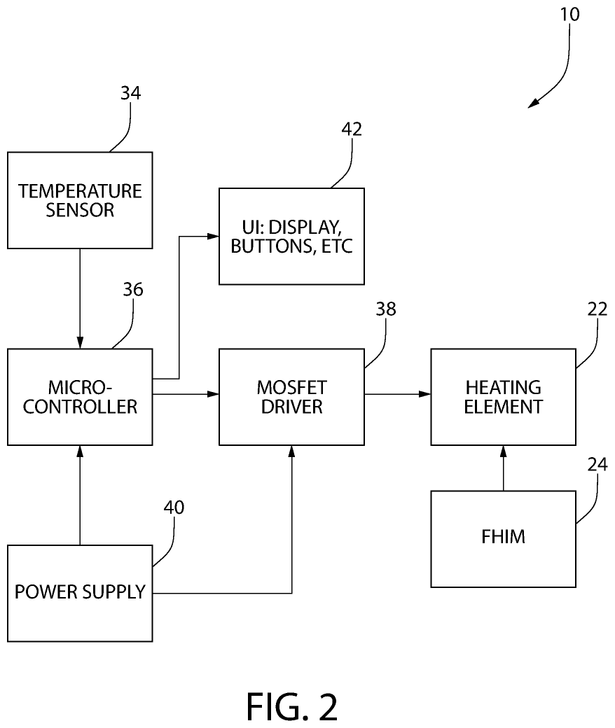

Referring now to the drawings wherein like reference numbers refer to like elements throughout the several views, there is shown in FIG. 2 an ocular surgery thermal control device 10 in accordance with an exemplary embodiment the present invention for use with an ocular surgery system 12 (see FIG. 1). The typical ocular surgery system 12 is, for example, a phacoemulsification system (for example, an Alcon Centurion® Vision System). The ocular surgery system 12 is used in an operating room setting as shown in FIG. 1 and may further include a fluid bottle 14 filled with, for example, balanced salt solution (BSS), an irrigation line 16, a phaco probe 18 and any other additional elements required to perform ocular surgery. The ocular surgery system 12 is intended for use on a patient 20.

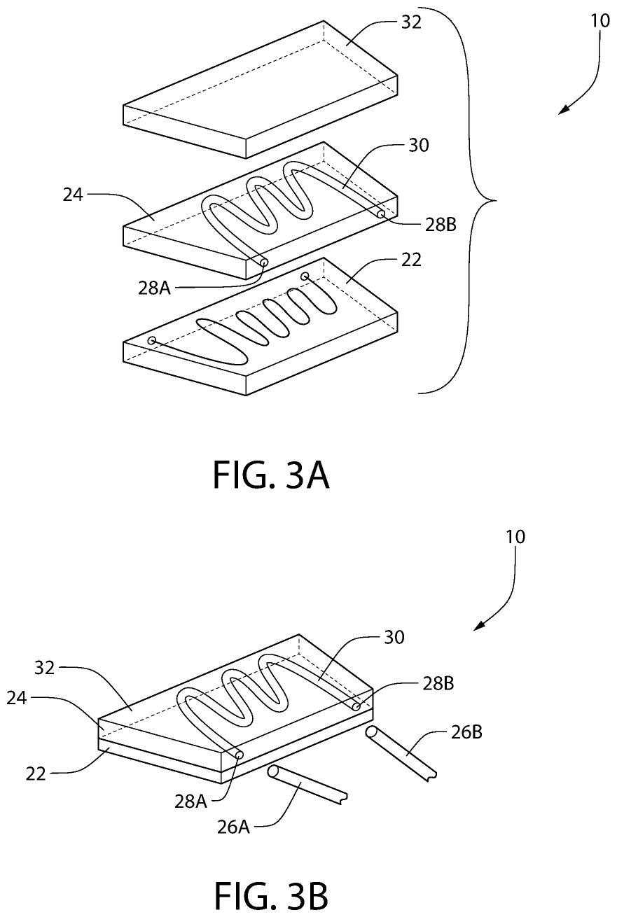

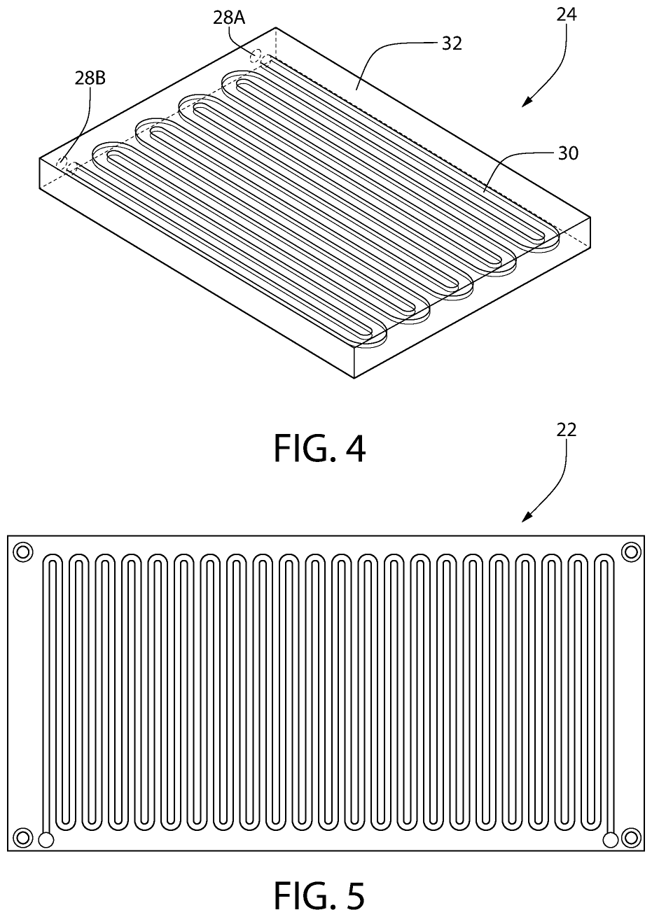

As shown in the block diagram of FIG. 2, the ocular surgery thermal control device 10 comprises a heating element 22 (see FIGS. 3A and 5), for example, a resistive heating element, interfaced with a fluid-heater interface module (FHIM) 24 (see FIGS. 3A, 3B and 4). The thermal control device 10 warms and cools the eye environment throughout an entire ocular surgical procedure, allowing surgeons to precisely change the temperature in real-time.

The ocular surgery thermal control device 10 is intended to be compatible with all models of surgical systems 12, for example, the phacoemulsification system, and is intended to work without disrupting surgical workflow. The thermal control device 10 is preferably insulated to ensure safe use for patients and OR staff, and is preferably in the form of a disposable cartridge that meets the strict sterility requirements of the OR.

As shown in FIG. 3B, the FHIM 16 interfaces with BSS tubing 26A, 26B via cold inflow port 28A and warm outflow port 28B. The BSS tubing 26A, 26B attaches to the FHIM 24, for example, via inflow port 28A and outflow port 28B on an exposed edge of the FHIM 24. The BSS is thereby warmed prior to injection into the eye of the patient 20. The FHIM 24 contains small winding heat transfer channels 30, allowing heat transfer to occur while BSS is flowing at, for example, up to 60 mL per minute. The heating element 22 has the capability to rapidly cool (for example, by removing power to the heating element), allowing surgeons the flexibility to adjust a target temperature in real time. The heating element 22, encapsulation 32, and user interface (described below) of the ocular surgery thermal control device 10 are reusable and sterilizable, while the FHIM 24 is disposable and compatible with current surgical setups and BSS tubing. The FHIM 24 can be easily removed and replaced between each surgery. The thermal control device 10 integrates, but does not interfere with, current features of the workflow such as fluidics and pressure control. The thermal control device 10 may be, for example, 8 inches×10 inches×8 inches, and is intended to be placed, for example, on a back table of the ocular surgery system 12 (for example, that found on Alcon phacoemulsification systems). A small size and proper insulation will prevent the device from interfering with the surgical technician's role.

As shown in the block diagram of FIG. 2, the ocular surgery thermal control device 10 includes several components. A temperature sensor 34 (that senses the temperature of BSS exiting the outflow port 28B) is connected to a microcontroller 36, which compares the current and desired temperature and outputs a signal to a MOSFET driver 38 in order to cause the heating element 22 to heat the BSS to the desired temperature. Preferably, the microcontroller 36 uses a proportional-integral-derivative (PID) control system, as known, to achieve fast and accurate temperature control. A power supply 38 provides power to the microcontroller 36 and the heating element 22 through the MOSFET driver 38. The device also has a user interface 42 where surgical technicians can see the current and desired temperature, and change the desired temperature on a display of the user interface.

For purposes of the present invention, the term “heating element” is intended to be broadly construed to be capable of any manner of heating and cooling. For example, the heating element can be a device (such as a heat exchanger) capable of either heating BSS (via, for example, a resistive heating element or thermoelectric device), and/or cooling (via, for example, a cooling source such as a refrigeration cycle).

The ocular surgery thermal control device 10 provides for surgical technicians to easily reset the device for the next procedure by replacing the used FHIM 24 with a new one. The thermal control device 10 is operated by surgical technicians and cataract surgeons, via the user interface 42, for example, via a display on its surface and control dials on its side. For example, one dial will control a current target temperature, while the screen will display both a output temperature and a current target temperature. The thermal control device 10 will warm BSS to physiological temperature, and lower temperature from physiological to room temperature.

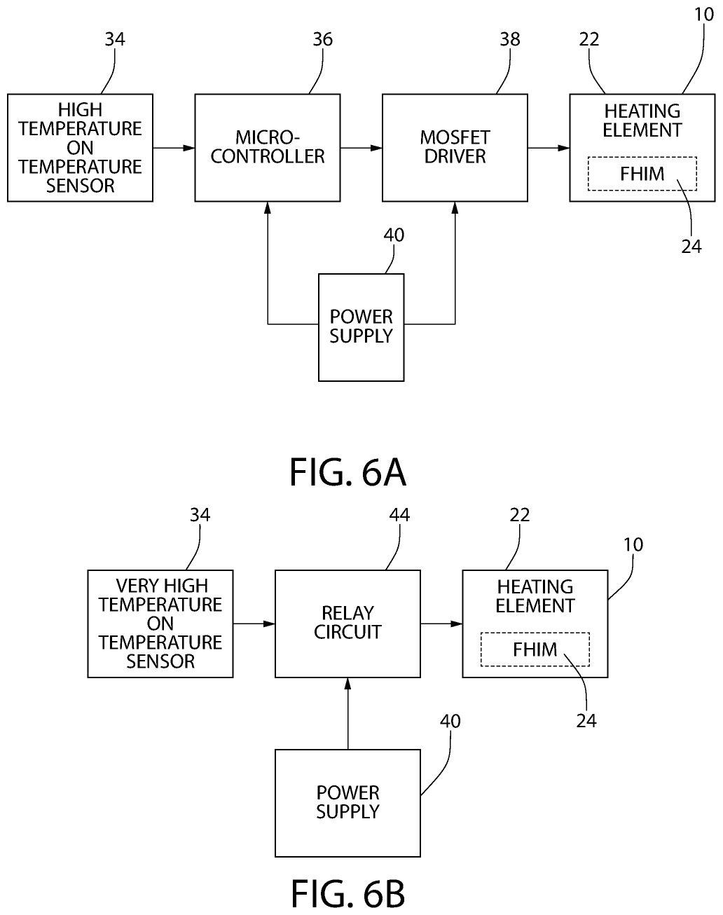

As can be seen in FIG. 6A, a primary failure mode for the thermal control device 10 of the present invention is that when overly high temperatures (e.g., a hardcoded threshold) are read by the temperature sensor 34, the microcontroller 36 sends a signal to the MOSFET driver 38 to cut power to the heating element 22. As can be seen in FIG. 6B, in case the microcontroller 36 fails, a backup failure mode is included in which the temperature sensor 34 is connected to a relay circuit 44. When the temperature sensor 34 senses a temperature at a certain desired threshold, the temperature sensor 34 causes the relay circuit 44 to close and prevent power from reaching the heating element 22.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. A thermal control device for an ocular surgery system, comprising:
(a) a fluid heater interface module (FHIM), the FHIM comprising:
(i) an inflow port for receiving balanced salt solution (BSS) from the ocular surgery system, and
(ii) an outflow port for dispensing the BSS to a probe of the ocular surgery system;
(iii) internal heat transfer channels between the inflow port and the outflow port;
(b) at least one heating element disposed adjacent to the FHIM to heat the heat transfer channels in the FHIM,
(c) at least one temperature sensor disposed adjacent the outflow port in the FHIM,
(d) a microcontroller to control heat output from the heating element, and
(e) a user interface comprising an outlet temperature control adjuster;
whereby dynamic, precise temperature control of portions of the eye via the BSS is obtained.

2. The thermal control device of claim 1, including a MOSFET driver to control the heating element.

3. The thermal control device of claim 1, including a power supply to provide power to the microcontroller.

4. The thermal control device of claim 2, including a power supply to provide power to the microcontroller and the MOSFET driver.

5. The thermal control device of claim 1, wherein the microcontroller provides proportional-integral-derivative (PID) control to the control temperature of BSS exiting the outflow port.

6. The thermal control device of claim 1, wherein the user interface provides for dynamic and adjustable temperature control by an ocular surgeon during ocular surgery

7. The thermal control device of claim 1, wherein temperature of BSS is displayed on the user interface in real time.

8. The thermal control device of claim 1, wherein the FHIM is designed for single use.

9. A method for thermal control of balanced salt solution (BSS) during ocular surgery, the method comprising:
(a) providing a fluid heater interface module (FHIM) comprising an inflow port and an outflow port, the inflow port for receiving balanced salt solution (BSS) from an ocular surgery system, an outflow port for dispensing the BSS to a probe of the ocular surgery system;
(b) heating the BSS by the providing power to a heating element to heat the FHIM;
(c) controlling a temperature of the BSS exiting the outlet port; and
(d) providing the controlled temperature BSS to an ocular surgery probe
whereby dynamic, precise temperature control of portions of the eye and the BSS is obtained.

10. The method for thermal control of claim 9, wherein the step of controlling the temperature of the BSS exiting the outlet port is controlled using proportion-integral-derivative control.
